(19) United States
(12) Patent Application Publication
(10) Pub. No.: US 2010/0069397 A1
(43) Pub. Date: Mar. 18, 2010

(54) SEXUAL DYSFUNCTION COMPOUNDS

(75) Inventors: Nils-Olof Lindberg, Malmo (SE); Katarina Lindell, Eslov (SE); Kristina Thyresson, Lund (SE); Alice C. Martino, Kalamazoo, MI (US)

Correspondence Address: FULBRIGHT & JAWORSKI, LLP, 1301 MCKINNEY, SUITE 5100, HOUSTON, TX, 77010-3095, US

(73) Assignee: Pharmacia AB

(21) Appl. No.: 12/623,723

(22) Filed: Nov. 23, 2009

Related U.S. Application Data
(63) Continuation of application No. 10/634,159, filed on Aug. 5, 2003.
(60) Provisional application No. 60/438,946, filed on Jan. 9, 2003.

(30) Foreign Application Priority Data
Aug. 5, 2002 (SE) 0202365-3

(51) Int. Cl. A61K 31/496; A61K 47/00; A61P 15/10

(52) U.S. Cl. 514/252.16; 514/783; 514/777; 514/771

(57) ABSTRACT

The present invention provides for a sexual dysfunction compound comprising a rapid-onset pharmaceutical composition that further comprises cocoa powder. The invention also provides for a process for manufacturing the composition, and use of the composition in sexual dysfunction therapy.

### CROSS-REFERENCE TO RELATED APPLICATIONS

This application claims priority to U.S. Provisional Application No. 60/438,946, filed Jan. 9, 2003, which is hereby incorporated by reference in its entirety herein, and to Swedish Application No. SE 0202365-3, filed Aug. 5, 2002, which is hereby incorporated by reference in its entirety herein.

### TECHNICAL FIELD

This invention relates to novel, rapid-onset, orally administered pharmaceutical compositions of sexual dysfunction (SD) compounds and the uses thereof. More particularly, the present invention relates to compositions comprising SD compounds and cocoa powder, methods to prepare the compositions, and to methods for using the compositions in sexual dysfunction therapy, including enhancement of sexual desire, and interest or performance.

### BACKGROUND OF THE INVENTION

Orally administered therapies for sexual dysfunction, in particular for male erectile disorder, are well known. See for example Gingell & Lockyer (1999), “Emerging pharmacological therapies for erectile dysfunction”, Expert Opinion on Therapeutic Patents 9, 1689-1696. Drugs in use or in development include phosphodiesterase type 5 (PDE5) inhibitors, e.g., sildenafil citrate, available under the trademark Viagra® of Pfizer, cyclic AMP activators, α-adrenergic antagonists, e.g., yohimbine, and dopaminergic agonists, e.g., apomorphine.

International Patent Publication No. WO 00/40226, incorporated herein by reference, discloses compounds useful in treating sexual dysfunction in men and women, these compounds being of formula (I)

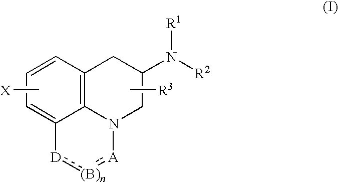

or pharmaceutically acceptable salts thereof, wherein R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;

X is H, F, Cl, Br, I, OH, C₁₋₆ alkyl or alkoxy, CN, carboxamide, carboxyl or (C₁₋₆ alkyl)carbonyl; A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N; B is CH, CH₂, CHF, CHCl, CHBr, CHI, C═O, N, NH or NCH₃, and n is 0 or 1; and D is CH, CH₂, CHF, CHCl, CHBr, CHI, C═O, O, N, NH or NCH₃; with various provisos indicated therein. WO 00/40226 further contemplates prescription of the drug (R)-5,6-dihydro-5-(methylamino)-4H-imidazo[4,5-ij]-quinolin-2(1H)-one (Z)-2-butenedioate (1:1) to male and female subjects at a dose of 1-3 mg, to be taken 0.5-1 h before engaging in sexual activity, and indicates that at such a dose and timing of administration the drug is therapeutically effective. No information is provided as to the route of administration or nature of dosage form.

The class of compounds proposed for treatment of sexual dysfunction in WO 00/40226 was earlier disclosed in U.S. Pat. No. 5,273,975 to Moon et al. to have therapeutically useful central nervous system activity. Certain compounds of the above class are the subject of a paper by Heier et al. (1997), “Synthesis and biological activities of (R)-5,6-dihydro-N,N-dimethyl-4H-imidazo[4,5,1-ij]quinolin-5-amine and its metabolites”, J. Med. Chem. 40, 639-646.

In spite of the availability of sildenafil citrate, apomorphine and other drugs in orally deliverable form, there remains a need for dosage forms of a therapeutic agent for treating sexual dysfunction in men and women, having one or more of the following benefits: (a) immediate absorption leading to rapid onset of therapeutic effect; (b) no requirement to be taken with water; (c) reduced unpleasantness of taste; (d) enhanced convenience of oral administration within a short interval prior to sexual activity; (e) discreet route of administration or method of use; and (f) low effective dose. Other needs also remain in the art.

In one aspect, sexual dysfunction as addressed herein comprises sexual disorders including, without limitation, hypoactive sexual desire disorder, female sexual arousal disorder, male erectile disorder, female orgasmic disorder and male orgasmic disorder, all as defined in Diagnostic and Statistical Manual of Mental Disorders, 4th edition (DSM-IV) (1994), and DSM-IV Guidebook (1995), both published by American Psychiatric Press, Inc., Washington, D.C.

In another aspect, sexual dysfunction as addressed herein comprises diminishment of sexual desire, interest and/or function arising from primary diseases or conditions that are not sexual disorders in a strict sense. Such diseases and conditions include, without limitation, epilepsy, craniopharyngioma, hypogonadism and general psychiatric disorders such as depression. Sexual dysfunction as addressed herein additionally comprises sexual deficiencies following hysterectomy and/or oophorectomy as well as those arising as side effects of medication.

European Patent Application No. 0 960 621 discloses that sildenafil citrate has an unpleasant taste that cannot be completely masked by flavoring agents, and proposes rapidly disintegrating oral dosage forms of sildenafil in the form of its free base, which has extremely low solubility in water and is virtually tasteless.

International Patent Publication No. WO 99/66933 proposes intranasal administration of sildenafil, illustratively in the form of salts such as the hydrochloride salt, for treatment of erectile dysfunction. Dosage forms proposed include a nasal spray and an aqueous nasal gel. Aqueous solutions are said to be preferred. Rapid onset of therapeutic effect is contemplated; however, no solution is suggested to the problem of unpleasant taste arising from drainage of the drug into the mouth. Further, intranasal administration is not a sufficiently discreet way of administering SD compounds. Dosage rates are contemplated in WO 99/66933 to be lower than are required when the drug is orally administered; a 30 mg dose of sildenafil hydrochloride in the form of a nasal spray is exemplified. Also exemplified is a nasal spray formulation delivering 30 mg of sildenafil hydrochloride and 1 mg of apomorphine hydrochloride.

European Patent Application No. 0 992 240 discloses cGMP-PDE inhibitory compounds said to be useful in treatment of male erectile dysfunction and proposes transmucomembranous administration, for example in the form of sublingual preparations, of such compounds.

International Patent Publication No. WO 00/76509 also proposes nasal administration of apomorphine, illustratively as its hydrochloride salt.

Heaton (1996), “Buccal apomorphine”, J. Urol. 155, 49, reports efficacy of a sublingual formulation of apomorphine in treatment of male non-organic erectile dysfunction.

U.S. Pat. No. 5,985,889 to El-Rashidy et al. proposes sublingual administration of apomorphine for treatment of male psychogenic erectile dysfunction. Various sublingual tablet formulations of apomorphine hydrochloride are disclosed therein.

International Patent Publication No. WO 00/35457, incorporated herein by reference, proposes use of apomorphine for treatment of male organic, e.g., vasculogenic, erectile dysfunction, and exemplifies use of a sublingual tablet formulation of apomorphine hydrochloride. WO 00/35457 further suggests that nausea, a common side effect of apomorphine, can be controlled by inclusion of an anti-emetic agent such as nicotine in the formulation.

U.S. Pat. No. 6,121,276 to El-Rashidy & Ronsen discloses flavored sublingual tablets containing apomorphine hydrochloride and nicotine.

International Patent Publication No. WO 01/49292 discloses sublingual tablets of apomorphine providing prolonged release of the drug, said to be useful in treatment of Parkinson's disease.

International Patent Publication No. WO 00/42992 discloses a dosage unit comprising a water-soluble hydrocolloid and sildenafil citrate in a mucoadhesive film said to be suitable for application to the oral mucosa. Pharmacokinetic data presented in WO 00/42992 indicate no faster absorption into the bloodstream with sublingual application of such a film than with a commercial tablet formulation of sildenafil citrate (Viagra®) at the same dosage.

International Patent Publication No. WO 01/10406 discloses compositions said to be suitable for a wide range of routes of administration of sildenafil citrate, including buccal and sublingual routes. Preferred compositions disclosed are said to comprise a solution, gel, semisolid, suspension, metered dose device, transdermal patch or film.

International Patent Publication No. WO 02/05820 discloses film dosage forms comprising sildenafil citrate. These dosage forms are prepared by mixing a solid dispersion of sildenafil citrate and a water soluble sugar with a hydrocolloid and optionally other ingredients, and are said, upon placement on a mucosal surface, to form a coating that subsequently disintegrates and dissolves to release sildenafil.

International Patent Publication No. WO 02/041840 discloses the use of cocoa powder as a flavourant, though not a taste-masker, in chewing gums for sildenafil citrate.

International Patent Publication No. WO 00/30641 discloses the use of cocoa powder as a flavourant in oral compositions containing nicotine.

International Patent Publication No. WO 99/66916 discloses the use of chocolate flavor in oral compositions containing apomorphine.

Nicotine replacement therapy is an established smoking cessation strategy. One problem with orally administered compounds for nicotine replacement therapy is that the absorption rate of nicotine is not rapid enough.

An attempt to solve the captioned problem is made with a nicotine-containing composition, preferably for buccal uptake, according to WO 00/30641. Herein is disclosed a composition comprising nicotine, at least one apolar component, at least one polar component and at least one surface-active component and optionally buffering agents. Anyhow, the composition according to WO 00/30641 has the disadvantage of insufficient taste masking of nicotine and buffering agents, and the drawback of causing nausea with some users. It should be noticed that WO 00/30641 does not disclose cocoa powder, neither as filler, diluent, taste-masking agent nor agent for providing a smooth texture.

Chocolate, which is very different from cocoa powder as such, is very rarely used as an ingredient in pharmaceutical products, hitherto only in laxatives. One example is Ex-Lax® being chocolated laxative pieces marketed by Novartis comprising sennosides. Purex, a laxative wherein phenolphthalein was formulated with chocolate, was marketed in the 1950s.

It has now surprisingly been found that a rapid onset of orally administered pharmaceutical compositions of SD compounds is achieved concomitantly with sufficient taste masking of badly tasting ingredients, such as buffering agents, through the use of SD-compound-containing formulations comprising cocoa powder as filler/diluent and taste masking or flavoring agent and agent for providing a smooth texture. No similar formulations have been disclosed hitherto.

### BRIEF SUMMARY OF THE INVENTION

An object of the invention is a sexual dysfunction compound-containing composition comprising cocoa powder as a vehicle, wherein the composition is orally administered for rapid onset. In a specific embodiment, the cocoa powder is at least 15%.

In a specific embodiment, the sexual-dysfunction-compound is selected from the group consisting of phosphodiesterase type 5 (PDE5) inhibitors, cyclic AMP activators, α-adrenergic antagonists, and dopaminergic agonists. In a further specific embodiment, the phosphodiesterase type 5 (PDE5) inhibitor is sildenafil or pharmaceutically acceptable salts of sildenafil. The sildenafil salt is sildenafil citrate in another specific embodiment.

In another specific embodiment of the invention, the phosphodiesterase type 5 (PDE5) inhibitor is tadalafil or vardenafil. In another embodiment, the α-adrenergic antagonist is yohimbine, phentolamine mesylate (e.g., Vasomex) or prasozin. In yet another embodiment, the dopaminergic agonist is apomorphine.

In one embodiment, the sexual-dysfunction-compound is the compound of formula (I)

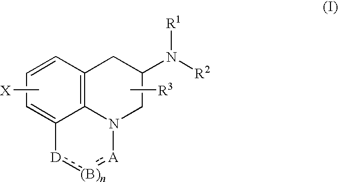

or a pharmaceutically acceptable salt thereof, wherein R¹, R² and R³ are the same or different and are selected from the group consisting of H, C₁₋₆ alkyl, C₃₋₅ alkenyl, C₃₋₅ alkynyl and C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups; X is selected from the group consisting of H, F, Cl, Br, I, OH, C₁₋₆ alkyl or alkoxy, CN, carboxamide, carboxyl and (C₁₋₆ alkyl)carbonyl; A is selected from the group consisting of CH, CH₂, CHF, CHCl, CHBr, CHI, CHCH₃, CO═O, C═S, CSCH₃, C═NH, CNH₂, CNHCH₃, CNHCOOCH₃, CNHCN, SO₂ or N; B is CH, CH2, CHF, CHCl, CHBr, CHI, C═O, N, NH or NCH₃, and n is 0 or 1; and D is CH, CH₂, CHF, CHCl, CHBr, CHI, C═O, O, N, NH or NCH₃; said compound of formula (I) or salt thereof being water-soluble.

In another embodiment of the invention, the sexual-dysfunction-compound is the compound of formula (II)

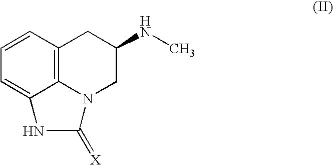

wherein X is O or S, and pharmaceutically acceptable salts thereof.

An embodiment of the invention is a sexual dysfunction compound-containing composition, further comprising one or more lipid ingredients. In a specific embodiment, the lipid is cocoa butter or cocoa butter alternatives. In a further specific embodiment, the cocoa butter alternatives are selected from the group consisting of cocoa butter equivalents (CBE), cocoa butter substitutes (CBS), cocoa butter replacers (CBR) and cocoa butter improvers (CBI). In one specific embodiment of the invention, the lipid is selected from the group consisting of oils based on lauric and myristic acids, oils based on palmitic, oleic and stearic acids, oils based on oleic, linoleic and linolenic acids, and oils based on animal fat. In an embodiment of the invention, the oils based on lauric and myristic acids are coconut oil or palmkernel oil.

In another embodiment of the invention, the oils based on palmitic, oleic and stearic acids are selected from the group consisting of palm oil, shea butter, karite butter, illipe butter, mango kernel oil, and sal fat. In yet another embodiment of the invention, the oils based on oleic, linoleic and linolenic acids are selected from the group consisting of corn oil, sunflower oil, hybrid sunflower oil, soybean oil, rapeseed oil, canola oil, olive oil, rice bran oil, cottonseed oil, peanut oil, and groundnut oil. The oils based on animal fat are fish oil, tallow, lard, or butterfat in another embodiment of the invention. In another embodiment of the invention, the lipid is a synthetic fat, reesterified fat, or hard fat.

One embodiment of the invention is sexual dysfunction compound-containing composition comprising cocoa powder as a vehicle, wherein the composition is orally administered for rapid onset further comprising one or more lipid ingredients and a buffering agent, wherein the buffering agent is selected from the group consisting of sodium carbonates, sodium bicarbonates, sodium phosphates, sodium glycinates, sodium acetates, sodium gluconates, sodium glycerophosphates, potassium carbonates, potassium bicarbonates, potassium phosphates, potassium glycinates, potassium acetates, potassium gluconates, potassium glycerophosphates, ammonium carbonates, ammonium bicarbonates, ammonium phosphates, ammonium glycinates, ammonium acetates, ammonium gluconates, ammonium glycerophosphates and mixtures thereof.

In one embodiment of the invention, the sexual dysfunction compound-containing composition further comprises at least one emulsifier/solubiliser. In a specific embodiment, the emulsifiers/solubilisers are selected from the group consisting of lecithin, a nonionic surfactant, an anionic surfactant, a zwitterionic surfactant and combinations with lecithin. In a further specific embodiment, lecithin is soy lecithin or egg lecithin. In one specific embodiment, the nonionic surfactant is selected from the group consisting of poloxamer, polyoxyethylene alkyl ether, polyoxyethylene castor oil derivative, polyoxyethylene sorbitan fatty acid ester, monoglyceride, diglyceride, polyoxyethylene stearate, polyglycerolester of fatty acids and sorbitan fatty acid ester. In another specific embodiment, wherein the anionic surfactant is selected from the group consisting of fatty acid, soap of fatty acid, lactylate, sodium lauryl sulfate and latanol. In a specific embodiment, the zwitterionic surfactant is a zwitterionic phospholipid.

In one embodiment of the invention, the sexual-dysfunction-compound-containing composition further comprises at least one sweetener, wherein the sweetener is selected from the group consisting of aspartame, acesulfame potassium, saccharine, cyclamate, glycyrrhizine, dihydrochalcones, stevisoide, thaumatin, monellin and neohesperidine.

In one embodiment of the invention, the sexual-dysfunction-compound-containing composition further comprises an amount of a substance/substances selected from the group consisting of fructose, glucose, galactose, lactose, maltose, invert sugar, polydextrose, a pharmaceutically acceptable polyol, and any mixture thereof. In a specific embodiment, the polyol is selected from the group consisting of xylitol, sorbitol, maltitol, mannitol, isomalt, glycerol, and any mixture thereof.

In one embodiment of the invention, the sexual-dysfunction-compound-containing composition further comprises a flavoring agent, wherein the flavoring agent is selected from the group consisting of peppermint, coffee, orange and vanilla.

An embodiment of the invention is a method for treating sexual dysfunction in a subject comprising the administration of a sexual dysfunction compound-containing composition as described herein in its various embodiments to said subject. In a specific embodiment, a unit dose comprises: a sexual-dysfunction-compound in a therapeutically effective amount; a diluent/filler; cocoa powder; an agent for providing a pharmaceutically acceptable polyol; a lipid ingredient; a buffering agent; a sweetener; an emulsifier/solubilizer; and a flavoring agent. In a specific embodiment, the polyol is from about 30% to about 70% (w/w).

In one embodiment of the invention, the unit dose comprises the lipid ingredient from about 30% to about 50% (w/w), the buffering agent from 0% to about 10% (w/w), the sweetener from about 0.3% to about 3% (w/w), the emulsifier solubilizer from about 0.3% to about 5% (w/w), and the flavoring agent from 0% to about 4% (w/w).

In one embodiment of the invention, the sexual-dysfunction-compound-containing composition further comprises fructose, glucose, galactose, or invert sugar.

An embodiment of the invention is a sexual-dysfunction-compound-containing orally administered rapid-onset pharmaceutical composition, wherein a unit dose thereof comprises from 0.25 mg to about 10 mg thereof of a compound of formula (II)

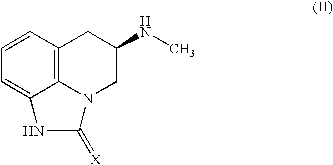

wherein X is O or S, and pharmaceutically acceptable salts thereof, about 50% (w/w) cocoa powder, about 44% (w/w) cocoa butter equivalents (CBE), about 4% (w/w) sodium carbonate, about 0.6% (w/w) aspartame and/or acesulfame potassium, and about 1% (w/w) lecithin.

An embodiment of the invention is a sexual-dysfunction-compound-containing orally administered rapid-onset pharmaceutical composition, wherein a unit dose thereof comprises a sexual-dysfunction compound according to formula (I)

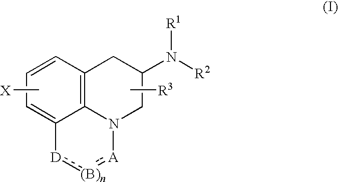

or a pharmaceutically acceptable salt thereof, in an amount from about 0.25 mg to around 10 mg, wherein R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups; X is H, F, Cl, Br, I, OH, C₁₋₆ alkyl or alkoxy, CN, carboxamide, carboxyl or (C₁₋₆ alkyl)carbonyl; A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N; B is CH, CH₂, CHF, CHCl, CHBr, CHI, C═O, N, NH or NCH₃, and n is 0 or 1; and D is CH, CH₂, CHF, CHCl, CHBr, CHI, C═O, O, N, NH or NCH₃; a diluent/filler; a flavoring/taste-masking agent; an agent for providing a smooth texture; a lipid ingredient; a buffering agent; a sweetener; an emulsifier/solubilizer; and a flavoring agent.

In a specific embodiment of the invention, the lipid ingredient is about 44% cocoa butter equivalents (CBE). In another specific embodiment, the buffering agent is about 4% sodium carbonate. In another specific embodiment, the sweetener is about 0.6% aspartame. In another specific embodiment, the emulsifier/solubilizer is about 1% lecithin. In another specific embodiment, the flavoring agent is about 0.5% mint or vanilla flavor. In another specific embodiment, the agent for providing a smooth texture is about 50% cocoa powder.

An embodiment of the invention is a sexual-dysfunction-compound-containing pharmaceutical composition which is formulated as an oral dosage form and which provides for delivery of sexual-dysfunction-compounds through the buccal mucosa and/or other mucosa of the oral cavity.

One embodiment of the invention is a method of manufacture of a sexual-dysfunction-compound-containing pharmaceutical composition as described herein in its various embodiments.

An embodiment of the invention is a method for treating sexual dysfunction comprising administration of a sexual-dysfunction-compound-containing pharmaceutical composition as described herein in its various embodiments to the subject less than 1 hour, or preferably less than 30 minutes prior to sexual activity.

### DETAILED DESCRIPTION OF THE INVENTION

### 1. Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The present invention provides an orally administered rapid-onset pharmaceutical composition useful for treatment of sexual dysfunction, stimulation of sexual activity and enhancement of sexual desire, interest and performance in men and women. The composition is a dosage form comprising a therapeutically or sexual-stimulatorily effective amount of one or more SD compounds. A sexual dysfunction compound is a compound appropriate for the treatment of sexual dysfunction disorders. A “therapeutically effective amount” herein is an amount sufficient to improve sexual desire, interest or performance in a subject having a sexual dysfunction condition. A “sexual-stimulatorily effective amount” herein is an amount sufficient to improve sexual desire, and interest or performance in a subject whether or not the subject has a sexual dysfunction condition.

The invention is adapted for discreet self-administration. By “discreet self-administration” herein is meant self-administration shortly prior to sexual activity in a way that does not draw attention of a sexual partner to, or emphasize, the existence of a sexual dysfunction, a need for therapy or a need or desire for enhancement of sexual performance. The combination of discreetness and rapid onset that is permitted by the present invention provides a benefit in spontaneity; by contrast, prior art compositions for treating sexual dysfunction can be seriously compromised in their effectiveness if their self-administration requires premeditation and/or cannot be done discreetly, such self-administration being thereby not conducive to spontaneity.

The term “taste-masking agent” used herein refers to an agent that is added to a composition to mask the taste of badly tasting components in the composition. For example, cocoa powder in the present invention masks the taste of the sexual dysfunction compound. Yet further, as used herein the terms “taste-masking agent” and “vehicle” are interchangeable.

The term “subject” as used herein, is taken to mean any mammalian subject to which a sexual dysfunction compound-containing composition is orally administered according to the methods described herein. In a specific embodiment, the methods of the present invention are employed to treat a human subject.

The term “sexual dysfunction compound-containing composition” as used herein refers to a composition, preferably a pharmaceutical composition containing at least one sexual dysfunction compound in a therapeutically or sexual-stimulatorily effective amount.

The term “buccal” as used herein is defined as for uptake buccally or by other mucosa in the oral cavity.

The term “transmucosal administration” or “transmucosal delivery” as used herein means any system or device for the administration of a drug across a subject's mucosal membrane, including the oral mucosa, such as the buccal and sublingual mucosa, and other mucosal membranes, including rectal, nasal, and vaginal. See “Controlled Drug Delivery, Fundamentals and Applications”, 2nd Ed., Robinson and Lee, eds., Chapter 1, “Influence of Drug Properties and Routes of Drug Administration on the Design of Sustained and Controlled Release Systems”, Li et al., Marcel Dekker Inc.: New York, pp. 3-61 (1987).

The term “disintegration” as used herein denotes melting, solubilization, erosion or a combinatorial effect of these physical changes of the invention.

The term “treating” and “treatment” as used herein refers to administering to a subject an effective amount of a “sexual dysfunction compound-containing composition so that the subject has an improvement in the disease or condition. The improvement is any improvement or remediation of the symptoms. The improvement is an observable or measurable improvement. Thus, one of skill in the art realizes that a treatment may improve the disease or condition, but may not be a complete cure for the disease or condition.

### 2. Sexual Dysfunction Compounds

Also provided by the present invention are methods of use of compositions of the present invention for treatment of sexual dysfunction and for enhancement of sexual desire, and interest or performance, and a method of use of a composition of the invention for preparing a medicament. Other features of this invention will be in part apparent and in part pointed out hereinafter.

It is the primary object of the present invention to provide rapid-onset pharmaceutical compositions useful for treatment of sexual dysfunction, stimulation of sexual activity and enhancement of sexual desire, interest or performance in men and women. The term “rapid-onset” means that a therapeutic effect is achieved within a short period of time, for example less than about 1 hour, preferably less than 30 minutes, following administration.

More specifically it is the object of the invention to provide such a SD-compound-containing composition, for transmucosal, preferably buccal, delivery, that disintegrates and/or melts at body temperature with or without the aid of salivary fluid or mechanical erosion, or a combination thereof after which the formulation preferably shows adhesiveness towards the tissues in the oral cavity.

Preferably, a dosage form of the invention is therapeutically effective when administered less than about 1 hour, more preferably less than 30 minutes, prior to sexual activity. Most preferred dosage forms of the invention are therapeutically effective when administered about 5 minutes to about 20 minutes, for example about 10 minutes to about 15 minutes, prior to sexual activity.

Suitable such SD compounds are chosen from the below agents, but are not limited thereto:

A compound of formula (I)

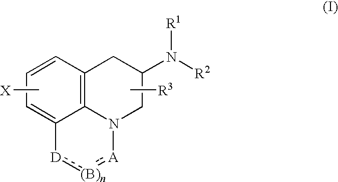

or a pharmaceutically acceptable salt thereof, wherein R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups; X is H, F, Cl, Br, I, OH, C₁₋₆ alkyl or alkoxy, CN, carboxamide, carboxyl or (C₁₋₆ alkyl)carbonyl; A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N; B is CH, CH₂, CHF, CHCl, CHBr, CHI, C═O, N, NH or NCH₃, and n is 0 or 1; and D is CH, CH₂, CHF, CHCl, CHBr, CHI, C═O, O, N, NH or NCH₃; said compound of formula (I) or salt thereof being water-soluble. A suitable dosing is from around 0.1 mg to around 10 mg per dose.

A compound of formula (II)

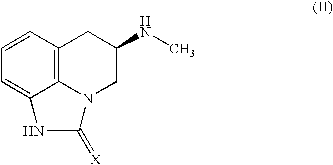

wherein X is O or S, and pharmaceutically acceptable salts thereof. A suitable dosing is from around 0.05 mg to around 10 mg per dose.

A compound chosen from phosphodiesterase type 5 (PDE5) inhibitors, cyclic AMP activators, noradrenergic alpha antagonists or α-adrenergic antagonists, and dopaminergic agonists. Examples of α-adrenergic antagonists include, but are not limited to phentolamine mesylate (e.g., Vasomax), yohimbine, and prasozin. Phosphodiesterases (PDEs) help control the intracellular levels of cGMP and cAMP. There are at least nine different types of PDEs. Papaverine is a nonselective PDE inhibitor that can be used to treat sexual dysfunction. Other suitable SD compounds are those targeted to specific PDEs. PDE5 inhibitors include sildenafil in base form and pharmaceutically acceptable salts thereof, including sildenafil citrate marketed under the trademark Viagra®. A suitable dosing is from around 5 mg to around 100 mg per dose. Also appropriate is tadalafil marketed as Cialis®. Suitable dosing is from around 5 mg to around 100 mg per dose. Another PDE5 inhibitors is vardenafil.

Dopaminergic agonists are appropriate for use in the invention. Apomorphine, with or without addition of anti-emetic agents is an example. Suitable dosing of apomorphine is from 0.5 mg to around 10 mg per dose.

It is preferred that the amount of the SD compound or salt thereof be lower than an amount causing significant side effects. A particularly useful dosage form of the present invention is a formulation that disintegrates or melts in the mouth without need for drinking water or other fluid.

Compositions for the therapeutic delivery of SD compounds are provided. Said compositions comprising SD compounds provide rapid transmucosal absorption in the oral cavity.

The SD compounds of the present invention include the parent forms as well as salts and complexes of the parent forms.

### 3. Pharmaceutical Compositions

An object of the invention is to provide new pharmaceutical compositions of SD compounds for buccal uptake or uptake by other mucosa in the oral cavity, especially such compositions comprising a large percentage of cocoa powder.

The main advantages provided by a composition according to the present invention are: it allows for rapid onset of the pharmacological effect; it provides for good taste masking properties due to the presence of cocoa powder; it does not require any water for swallowing; it provides for possible high bioavailability for substances with high first pass metabolism; it provides for an association of pleasure; and it does not give an immediate patient-perceived association with medicines (traditional tablets).

The addition of buffering agents provides for a transient change in local pH of the saliva. Thereby a higher fraction of the active agent is transformed into its less ionized form. Thereupon the transmucosal permeation is facilitated, which enhances the absorption of the active agent. For those skilled in the art it is evident that the choice of the buffering system is dependent on the one or more pKas of the active agent.

It has surprisingly been found that a rapid buccal absorption of SD compounds concomitantly with sufficient taste masking of badly tasting ingredients, such as the active compound and/or buffering agents, is achieved through the use of cocoa powder. The cocoa powder acts as filler/diluent as well as taste masking or flavoring agent and agent for providing a smooth texture. No similar formulations have been disclosed hitherto.

A preferred formulation is a composition, weighing around 400 mg-500 mg, having the following ingredients: a therapeutically efficient amount of a SD compound; cocoa powder around 200 mg; fatty components around 180 mg; aspartame around 2.5 mg; sodium carbonate around 15 mg; and lecithin around 4 mg.

Cocoa powder is defined as cocoa nib with some fat removed and ground into a powder. Cocoa nib is defined as cocoa beans with the shell removed. Cocoa butter is defined as fat expelled from the center (kernels or nib) of cocoa beans.

Cocoa powder is prepared from roasted cocoa beans. It is a complex compound, which consists of starch, cocoa butter, amino acids, proteins, xanthines, amines, mono- and polysaccharides, phospholipids, flavonoids, pyrazines, etc.

Preferred fatty components are fats/lipids chosen from tempering fats, including cocoa butter equivalents (CBE) and cocoa butter improvers (CBI), and non-tempering fats, including cocoa butter replacers (CBR) and cocoa butter substitutes (CBS).

Chocolate is defined as a product obtained from cocoa nib, cocoa mass powder and sucrose with or without added cocoa butter, having a minimum dry cocoa solids content of 35%, at least 14% of dry non-fat cocoa solids and 18% cocoa butter. Chocolate has two major distinguishing characteristics: its flavor and its texture. A primary feature of the texture is that the chocolate must be solid at a temperature of 20-25° C. and yet melt rapidly in the mouth at 37° C. thereby being transferred to a liquid, which appears smooth to the tongue. The processing of chocolate is related to obtaining these two criteria (Industrial Chocolate Manufacture and Use, S. T. Beckett, ed., 2nd edition, Blackier Academic & Professional, London, 1994, p 382).

Neither milk chocolate nor light cooking chocolate or dark cooking chocolate may mask the disagreeable taste of most buffering agents. The cocoa content of milk chocolate is comparatively low (a cocoa mass content of 10-16%, corresponding to approximately 5-8% cocoa powder). The beans'/cocoa mass' content of dark, bittersweet chocolate is 55-70% (Beckett, pp. 276-277), corresponding to approximately 28-35% cocoa powder. By making a vehicle with a high proportion of cocoa powder (30-70%) and fatty components (30-50%), as per the present invention, an effective masking is though obtained. The higher the cocoa powder concentration the better the taste masking.

Other useful embodiments of the present invention are obtained by exchanging some of the above-mentioned excipients for equivalently functioning alternative compounds. For example, a small part of the cocoa powder, acting as diluent/filler and taste-masking/smoothening/flavoring agent, may be exchanged for one or more of the compounds sucrose, fructose, glucose, galactose, lactose, maltose, invert sugar, a pharmaceutically acceptable polyol such as xylitol, sorbitol, maltitol, mannitol, isomalt and glycerol, or polydextrose, or any mixture thereof, but only to such an extent that the taste-masking effect of the cocoa-powder remains sufficient.

The lipid ingredient of the present invention, being fatty components, may be chosen from one or more of the following compounds: cocoa butter and cocoa butter alternatives (i.e., cocoa butter equivalents (CBE), cocoa butter substitutes (CBS), cocoa butter replacers (CBR) and cocoa butter improvers (CBI)); coconut, palmkernel oil and other similar oils (other similar oils include oils that are characterized by being predominantly based on lauric and myristic acids; palm oil, shea butter, karite butter, illipe butter, mango kernel oil, sal fat and other similar fats (other similar fats include fats that are characterized by being predominantly based on palmitic, oleic and stearic acids); corn oil, sunflower oil, hybrid sunflower oil, soybean oil, rapeseed oil, canola oil, olive oil, rice bran oil, cottonseed oil, arachis (peanut, groundnut) oil and other oils (other oils include oils that are characterized by being predominantly based on oleic, linoleic and linolenic acids and hydrogenated to a suitable melting point); fish oil, tallow, lard, butterfat and other animal derived fats; and synthetic fats, reesterified fats, hard fats obtained by a chemical reaction of fatty acids with glycerol using no, acidic, alkaline or enzymatic catalysis. The compounds can be used as a single component or mixed with each other, being either crude or refined using physical or alkaline refining, or being subjected to further processing including catalytic hydrogenation, interesterification, transesterification and fractionation. Preferred fatty components are fats/lipids chosen from tempering fats, including cocoa butter equivalents (CBE) and cocoa butter improvers (CBI), and non-tempering fats, including cocoa butter replacers (CBR) and cocoa butter substitutes (CBS).

The buffer sodium carbonate may be exchanged for carbonates, bicarbonates, acetates, gluconates, glycerophosphates, phosphates, glycinates, citrates, malates and/or tartrates of sodium, potassium or ammonium, or mixtures thereof. Most phosphates are though less suitable because their taste usually is disagreeable and difficult to mask.

The sweetener aspartame may entirely or in part be exchanged for one or more other artificial sweeteners, such as acesulfame potassium, saccharine, cyclamate, glycyrrhizine, dihydrochalcones, stevioside, thaumatin, monellin and/or neohesperidine.

The emulsifier lecithin is preferably soy lecithin and/or egg lecithin, but may be exchanged for a nonionic surfactant (i.e., poloxamer, polyoxyethylene alkyl ether, polyoxyethylene castor oil derivative, polyoxyethylene sorbitan fatty acid ester, mono-glyceride, diglyceride and esther thereof, polyoxyethylene stearate, polyglycerolester of fatty acids (including polyglycerolpolyricinoleic acid (PGPR)), sorbitan fatty acid ester); an anionic surfactant (i.e., fatty acid, soap of fatty acid, lactylate, especially sodium and/or calcium stearoyllactylate, sodium lauryl sulfate and latanol); a zwitterionic surfactant (i.e., zwitterionic phospholipid, such as phosphati-dylcholine and phosphatidylethanolamine) or mixtures, fractions or derivatives thereof or with lecithin.

If cocoa mass comprising phospholipids is used instead of part of the cocoa powder the emulsifier/solubilizer may be removed from the composition.

Optionally, liquid or solid flavoring agents may be added. Non-limiting examples of flavoring agents are peppermint, coffee, orange and vanilla.

### EXAMPLES

Below follows non-limiting examples on preparation of certain embodiments of the present invention.

### Example 1

### Preparation of an SD Compound-Containing Composition

A composition weighing around 400 mg is manufactured, having the following preferred composition (w/w):

The active ingredient is an SD compound according to previously described formula (I) in an amount from around 0.25 mg to around 10 mg. Additionally, the composition comprises a diluent/filler, a flavoring/taste-masking agent, and agent for providing a smooth texture. Specifically, the smoothing agent is cocoa powder around 50%. The composition also comprises a lipid ingredient, specifically cocoa butter equivalents (CBE) around 44%. Also included are a buffering agent, sodium carbonate around 4%, a sweetener, aspartame around 0.6%, and an emulsifier/solubilizer, lecithin around 1%.

The flavoring agent, a mint or vanilla flavor 0.5%, is prepared as follows. A part of the CBE is melted. The solid components, which include the SD compound, cocoa powder, aspartame, sodium carbonate and the flavoring agent if solid, are added and mixed. A reduction of particle size of the solid components is performed by milling in a roll-refiner. If the solid components have already achieved the required particle size, e.g. by milling before the mixing with the fatty components, roll refining is dispensed with. After treatment in the roll-refiner, the mixture is mixed with the rest of the melted fatty components or remelted (if solidified) and mixed with the rest of the melted CBE. A mixing of the melt is performed in a suitable mixer. The liquid components, i.e. lecithin and the flavoring agent if liquid, are added. Tablets or other solid dosage forms are subsequently made using suitable techniques, such as molding, extrusion or congealing, including pastillation, when necessary after suitable preconditioning. Also other suitable manufacturing methods, which are known to a skilled artisan, may be used.

### Example 2

### Preparation of an an SD Compound-Containing Composition Comprising Formula II

In essentially the same way as in Example 1, a composition with a weight from around 400 mg to around 500 mg having the below ingredients is manufactured.

Included in the composition are a compound as defined by formula (II), in the amount from 0.25 mg to around 10 mg, around 50% (w/w) cocoa powder, around 44% (w/w) cocoa butter equivalents (CBE), around 4% (w/w) sodium carbonate, around 0.6% (w/w) aspartame and/or acesulfame potassium, and around 1% (w/w) lecithin.

### Example 3

### Preparation of Compositions Using SD Compounds

In essentially the same way as in Example 1 a composition with the below contents is manufactured.

The active component is an SD compound in a therapeutically sufficient amount.

The diluent/filler component is as described. The composition further includes cocoa powder and optionally a small amount of a flavoring/taste substance or substances chosen from one or more of the following: a masking agent, fructose, glucose, galactose, or invert sugar. Also the composition includes an agent for providing a pharmaceutically acceptable polyol such as xylitol, and an agent for providing a smooth texture, such as sorbitol, maltitol, mannitol, isomalt and glycerol, or polydextrose, or any mixture thereof, from around 30% to around 70% (w/w). Further the composition includes a lipid ingredient from around 30% to around 50% (w/w), a buffering agent from 0% to around 10% (w/w), a sweetener from around 0.3% to around 3% (w/w), an emulsifier/solubilizer from around 0.3% to around 5% (w/w), and a flavoring agent from 0% to around 4% (w/w).

### Example 4

### Preparations of Compositions with Excipients

Useful compositions are obtained by exchanging some of the excipients in the compositions of the above examples for equivalently functioning alternative compounds.

A small part of the cocoa powder may be exchanged for one or more of the compounds fructose, glucose, galactose, lactose, maltose, invert sugar, a pharmaceutically acceptable polyol such as xylitol, sorbitol, maltitol, mannitol, isomalt and glycerol, or polydextrose, or any mixture thereof, but only to such an extent that the taste-masking effect of the cocoa-powder remains sufficient.

The lipid ingredient, being fatty components, may be chosen from one or more of the following compounds: (1) cocoa butter and cocoa butter alternatives, including cocoa butter equivalents (CBE), cocoa butter substitutes (CBS), cocoa butter replacers (CBR) and cocoa butter improvers (CBI); (2) coconut, palmkernel oil and other similar oils characterized by being predominantly based on lauric and myristic acids; (3) palm oil, shea butter, karite butter, illipe butter, mango kernel oil, sal fat and other similar fats characterized by being predominantly based on palmitic, oleic and stearic acids; (4) corn oil, sunflower oil, hybrid sunflower oil, soybean oil, rapeseed oil, canola oil, olive oil, ricebran oil, cottonseed oil, arachis (peanut, groundnut) oil and other oils characterized by being predominantly based on oleic, linoleic and linolenic acids and hydrogenated to a suitable melting point; (5) fish oil, tallow, lard, butterfat and other animal derived fats; and (6) synthetic fats, reesterified fats, hard fats obtained by a chemical reaction of fatty acids with glycerol using no, acidic, alkaline or enzymatic catalysis, whereby said compound(s) is/are used as a single component or mixed with each other, being either crude or refined using physical or alkaline refining, or being subjected to further processing including catalytic hydrogenation, interesterification, transesterification and fractionation.

The buffer sodium carbonate may be exchanged for carbonates, bicarbonates, acetates, gluconates, glycerophosphates, phosphates or glycinates of sodium, potassium or ammonium, or mixtures thereof. Most phosphates are though less suitable because their taste usually is disagreeable and difficult to mask.

The sweetener aspartame may entirely or in part be exchanged for one or more other artificial sweeteners, such as acesulfame potassium, saccharine, sodium saccharine, cyclamate and glycyrrhizine and/or salts thereof.

The emulsifier lecithin is preferably soy lecithin and/or egg lecithin, but may be exchanged for (1) a nonionic surfactant, such as poloxamer, polyoxyethylene alkyl ether, poly-oxyethylene castor oil derivative, polyoxyethylene sorbitan fatty acid ester, mono-glyceride, diglyceride and esther thereof, polyoxyethylene stearate, polyglycerolester of fatty acids (including polyglycerolpolyricinoleic acid (PGPR)), sorbitan fatty acid ester; (2) an anionic surfactant, such as fatty acid, soap of fatty acid, lactylate, especially sodium and/or calcium stearoyllactylate, sodium lauryl sulfate and latanol; (3) a zwitterionic surfactant, such as zwitterionic phospholipid, such as phosphati-dylcholine and phosphatidylethanolamine; or mixtures, fractions or derivatives thereof or with lecithin.

In principally the same way as in the above examples compositions comprising other SD compounds may be manufactured. The dose range and the percentages of the excipients should in such cases be accordingly adjusted.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.
Non-Patent Publications:
“Controlled Drug Delivery, Fundamentals and Applications”, 2nd Ed., Robinson and Lee, eds., Chapter 1, “Influence of Drug Properties and Routes of Drug Administration on the Design of Sustained and Controlled Release Systems”, Li et al., Marcel Dekker Inc.: New York, pp. 3-61 (1987).
Gingell & Lockyer (1999), “Emerging pharmacological therapies for erectile dysfunction”, Expert Opinion on Therapeutic Patents 9, 1689-1696.
Heaton (1996), “Buccal apomorphine”, J. Urol. 155, 49, reports efficacy of a sublingual formulation of apomorphine in treatment of male non-organic erectile dysfunction.
Heier et al. (1997), “Synthesis and biological activities of (R)-5,6-dihydro-N,N-dimethyl-4H-imidazo[4,5,1-ij]quinolin-5-amine and its metabolites”, J. Med. Chem. 40, 639-646.
Industrial Chocolate Manufacture and Use, S. T. Beckett, ed., 2nd edition, Blackier Academic & Professional, London, 1994, p 382

### International Patents and Patent Application Publications:

### European Patent Application No. 0 960 621

### European Patent Application No. 0 992 240

### WO 00/40226

### WO 99/66933

### WO 00/76509

### WO 00/35457

### WO 01/49292

### WO 00/42992

### WO 01/10406

### WO 02/05820

### WO 00/30641

### WO 02/041840

### WO 99/66916

### U.S. Patents:

U.S. Pat. No. 6,121,276
U.S. Pat. No. 5,273,975
U.S. Pat. No. 5,985,889

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

1-48. (canceled)

49. A sexual dysfunction compound-containing composition that does not comprise a chewing gum;
comprising a buffering agent, at least one lipid ingredient, at least one emulsifier/solubiliser, and at least 50% cocoa powder;
wherein the cocoa powder is both a vehicle and taste-masking agent;
wherein the cocoa powder is not present as an ingredient in chocolate;
wherein the lipid is cocoa butter or a cocoa butter alternative selected from the group consisting of cocoa butter equivalents (CBE), cocoa butter substitutes (CBS), cocoa butter replacers (CBR) and cocoa butter improvers (CBI);
wherein the composition is capable of being orally administered for immediate absorption leading to rapid onset of therapeutic effect; and
wherein the composition disintegrates and/or melts at body temperature without the aid of salivary fluid or mechanical erosion or a combination thereof.

50. The sexual dysfunction compound-containing composition of claim 49, further comprising a flavoring agent selected from the group consisting of peppermint, coffee, orange, and vanilla.

51. The sexual dysfunction compound-containing composition of claim 49, further comprising at least one sweetener selected from the group consisting of aspartame, acesulfame potassium, saccharine, cyclamate, glycyrrhizine, dihydrochalcones, stevisoide, thaumatin, monellin and neohesperidine.

52. The sexual dysfunction compound-containing composition of claim 51 further comprising one or more of the substance/substances selected from the group consisting of fructose, glucose, galactose, lactose, maltose, invert sugar, polydextrose, a pharmaceutically acceptable polyol, and any mixture thereof.

53. The sexual dysfunction compound-containing composition of claim 52, wherein the polyol is selected from the group consisting of xylitol, sorbitol, maltitol, mannitol, glycerol, and isomalt.

54. The sexual dysfunction compound-containing composition of claim 49, wherein the buffering agent is selected from the group consisting of sodium carbonates, sodium bicarbonates, sodium phosphates, sodium glycinates, sodium acetates, sodium gluconates, sodium glycerophosphates, potassium carbonates, potassium bicarbonates, potassium phosphates, potassium glycinates, potassium acetates, potassium gluconates, potassium glycerophosphates, ammonium carbonates, ammonium bicarbonates, ammonium phosphates, ammonium glycinates, ammonium acetates, ammonium gluconates, ammonium glycerophosphates and mixtures thereof.

55. The sexual dysfunction compound-containing composition of claim 49, wherein the sexual-dysfunction-compound is selected from the group consisting of phosphodiesterase type 5 (PDE5) inhibitors, cyclic AMP activators, α-adrenergic antagonists, and dopaminergic agonists.

56. The sexual dysfunction compound-containing composition of claim 55 wherein the phosphodiesterase type 5 (PDE5) inhibitor is sildenafil or pharmaceutically acceptable salts of sildenafil.

57. The sexual dysfunction compound-containing composition of claim 56, wherein the pharmaceutically acceptable salts of sildenafi is sildenafil citrate.

58. The sexual dysfunction compound-containing composition of claim 49, wherein the emulsifiers/solubilisers are selected from the group consisting of lecithin, a nonionic surfactant, an anionic surfactant, a zwitterionic surfactant and combinations with lecithin, wherein the lecithin is soy lecithin or egg lecithin.

59. The sexual dysfunction compound-containing composition of claim 58, wherein the nonionic surfactant is selected from the group consisting of poloxamer, polyoxyethylene alkyl ether, polyoxyethylene castor oil derivative, polyoxyethylene sorbitan fatty acid ester, monoglyceride, diglyceride, polyoxyethylene stearate, polyglycerolester of fatty acids and sorbitan fatty acid ester.

60. The sexual dysfunction compound-containing composition of claim 58, wherein the anionic surfactant is selected from the group consisting of fatty acid, soap of fatty acid, lactylate, sodium lauryl sulfate and latanol.

61. The sexual dysfunction compound-containing composition of claim 58, wherein the zwitterionic surfactant is a zwitterionic phospholipid.

62. The sexual dysfunction compound-containing composition of claim 49, wherein the composition does not contain sucrose.
